(19) United States
(12) Patent Application Publication
(10) Pub. No.: US 2014/0371639 A1
(43) Pub. Date: Dec. 18, 2014

(54) Thermal Therapy Massage Roller

(71) Applicant: Christopher Diecidue, Fort Lauderdale, FL (US)

(72) Inventor: Christopher Diecidue, Fort Lauderdale, FL (US)

(21) Appl. No.: 14/287,876

(22) Filed: May 27, 2014

Related U.S. Application Data
(60) Provisional application No. 61/827,187, filed on May 24, 2013.

(51) Int. Cl. A61H 15/00; A61F 7/00

(52) U.S. Cl. CPC A61H 15/00; A61F 7/00; A61H 2201/0207
USPC 601/19

(57) ABSTRACT

A thermal therapy massage roller includes an elongate substantially cylindrical rigid sleeve defining an elongate cavity proportioned for receipt of a rechargeable battery having an electrode and charging adaptor at a base of the battery; a layer of insulation secured about the rigid sleeve; a circumferential heating element secured about the layer of insulation, the heating element in electrical communication with the battery; a layer of flexible insulative material surrounding the heating element, and an outer layer of a resilient and flexible foam-like material circumferentially surrounding the insulative layer, the foam-like material defining an outer layer of the roller.

### REFERENCE TO RELATED APPLICATION

This application claims the benefit under 35 USC 119(e) of provisional patent Application Ser. No. 61/827,187, filed May 24, 2013, which is incorporated by reference in its entirety.

### BACKGROUND OF THE INVENTION

1. Field of the Invention

This invention relates to massage implements and, more particularly, to a thermal therapeutic massage for applying rolling pressure to fascia, muscles, ligaments, tendons and joints.

2. Description of the Related Art

The present invention constitutes an improvement to standard massage devices otherwise well-known and commonly available in the art. Massage implements, as are known, primarily include grippable unitary objects for use by a massage therapist in deep tissue massage such as shiatsu, Bowen therapy, Balinese massage and the like. Massage implements of this type to serve the common goals of relieving muscle aches and sore muscles, promoting blood circulation, relieving muscle cramps, loosing tight and knotted muscles, and relieving pressure upon the massage therapist hands. These implements run the gamut from the pulling and pushing of tissues to a spectrum of articles including objects as simple as heated stones to as complex as hand held tools with complexly formed protrusions for the kneading of fascia and other such connective tissue. These objects are typically not intended for self-use by a person suffering from sore muscles, joints or the like and, often, only contact small surface areas of tissue and are not adapted for the application of pressure to multiple sites or across a larger surface area that may require attention. Another issue in the prior art is the existence of a high friction which may result between the tissue and the tool as well as a lack of an evenness of pressure applied to the tissues in the areas of the massage as the object is moved across the tissue.

Few such articles, tools or devices in the prior art are readily rollable across the subject area of treatment or make use of foam components thereof to enable evenness in the application of pressure as needed for the proficient practice of certain massage techniques and, also, for enhanced comfort to otherwise sensitive areas of the body of the patient.

The prior art in the present area is represented by U.S. Application Publication No. US 2012/031025 to Hall, entitled Cylindrical Roller Massage Tool. Hall is a simple cylindrical massage tool having fixed handles at opposite ends thereof and a foam-like cylindrical component which rotates upon an inner axle which is journalled within the handles of the system.

U.S. Patent Application Publication No. 2012/0323151 to Fausset, entitled Rolling Muscle Massager is conceptually, similar to Hall, however is provided with a circumferential layer which may be chilled in a refrigerator or freezer or heated by a microwave such that the skin contact portion, when wrapped around a spindle element, may provide a cooling or heating capability to the user. As such, Hall contemplates a system which may be employed either for the massaging of one's own muscle or for the muscles or tissue of another individual or partner.

U.S. Patent Application Publication No. 2013/0012848 to Forrest teaches a massage device including both heating and vibratory elements which is also provided with an internal fan to facilitate air flow through openings in a circumferential sleeve thereof and to provide a therapeutic effect to the patient.

U.S. Patent Application Publication No. 2012/0322633 to Holman, entitled Exercise Roller with Resistance Bands teaches a resistance band body roller which may be provided with a rubber or foam-like surface upon which the feet of the user would typically be applied while the resistance bands of Holman are pulled by the arm of the user to thereby simulate a rowing-like effect.

U.S. Patent Application No. 2010/0274165 to Evans, entitled Multi-Roller

Massage Device teaches a massage system in which each of the rollers thereof are provided with a soft external layer which may include a foam. Evans does not teach a heating or cooling function associated therewith.

The present invention thereby seeks to provide a thermal therapy massage roller having the comfort aspect associated with foam-like devices and the therapeutic benefits associated with heating and vibratory massage systems.

### SUMMARY OF THE INVENTION

The present invention is directed to a thermal therapy massage roller which includes an elongate substantially cylindrical thermally conductive body having an insulated elongate cavity therein proportioned for receipt of a rechargeable battery and having an electrode interfacable with a charging and plug adaptor from an AC power supply. The massage roller also includes a layer of insulation secured about said cylindrical body, outwardly of said battery. Further included is a pad-like circumferential heating element secured about said layer of insulation, said heating element in electrical communication with said battery with a layer of flexible insulative material surrounding the heating element. The massage roller yet further includes a layer of resilient and foam-like material circumferentially surrounding said insulative material to define an outer layer of the roller. Further insulative layers may be included between said heating element and said outer layer of foam-like material. Through the use of such material through which the heat of the heating may only partially pass, therapeutic properties of the heat operate synergistically with the resilient properties of the outer foam-like layer to provide a pleasing and beneficial effect to the user.

It is accordingly an object of the present invention to provide a thermal massage therapy device which while providing comfort to the skin and tissue of the user to provide a beneficial degree of heat to the affected tissue.

It is a further object to provide a thermal therapy device which may operate without need to microwave any operative portion thereof before its use.

It is another object of the invention to provide a thermal massage therapy device which permits the application of considerable pressure without the risk of injury to the patient as would be the case in the absence of the foam-like surface of the present device and its associated thermal capacity.

It is a yet further object to provide a device effective in the producing of myo-fascsial release and reduction of pain.

It is another object of the invention to provide a device of the above type which can be used either by an individual or as under the direction of a qualified healthcare professional or personal trainer.

The above and yet further objects and advantages of the present invention will become apparent from the hereinafter set forth Brief Description of the Drawings, Detailed Description of the Invention and Claims appended herewith.

### BRIEF DESCRIPTION OF THE DRAWINGS

FIG. 1 is a conceptual view of the inventive device showing the location of the rechargeable battery thereof.

FIG. 2 is a radial cross-sectional view of a first embodiment taken along Line 2-2 of FIG. 1.

FIG. 3 is a radial cross-sectional view of a second embodiment taken along Line 3-3 of FIG. 1.

FIG. 4 is a view of a third embodiment of the invention.

FIG. 5 is a view of a further embodiment of the invention of FIG. 4.

### DETAILED DESCRIPTION OF THE INVENTION

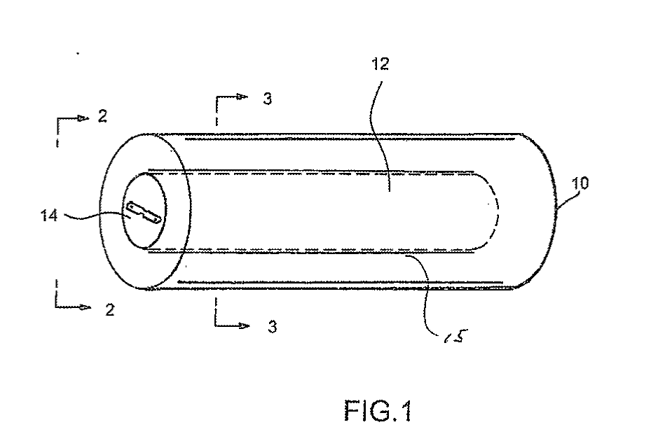

With reference to FIG. 1, there is shown a perspective external view of the contemplated thermal therapy massage roller 10, the power source of which is, in a first embodiment, a rechargeable battery 12 having a receptacle 14 at one electrode thereof. Receptacle 14 is preferably defined by a rigid insulative sleeve 15 to protect the battery from heat of the roller.

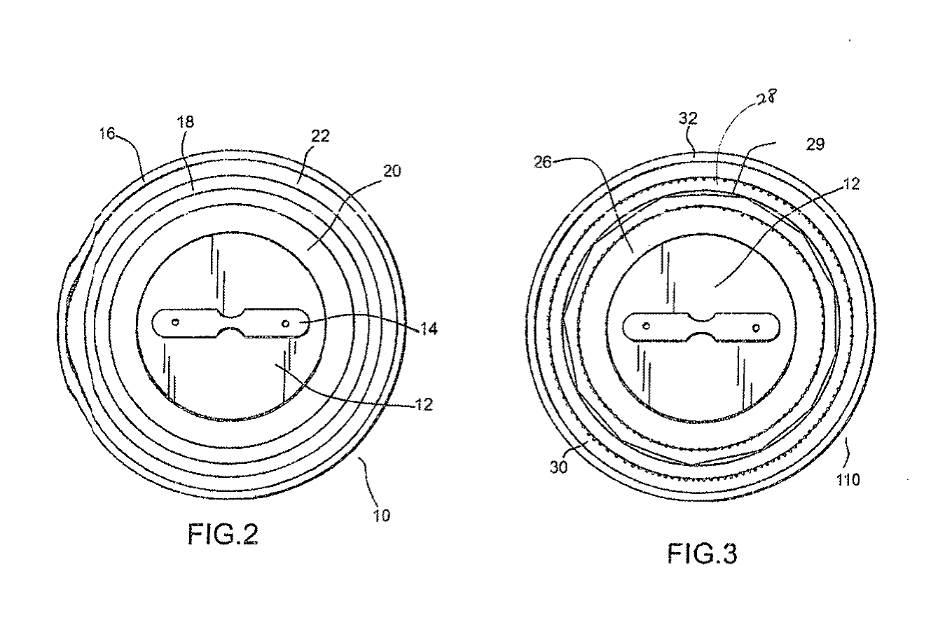

FIG. 2 is a conceptual axial end view taken along Line 2-2 of FIG. 1. Therein may be seen said receptacle 14 of rechargeable battery 12 and, additionally, may be seen an outer layer of foam 16 within which may also be a second layer of foam 18 as well as, optionally, a third layer of foam 20. Outwardly of innermost foam layer 20 is disposed a heating pad 24 having therein a resistive heating element which is in electrical communication with the electrodes of battery 12. Outwardly thereof is said foam layer 18, said insulative layer 22 and the outermost foam layer 16. It is to be understood that the intermediate foam layer 18 is optional in the embodiment of FIG. 2.

In the central radial cross-sectional view of FIG. 3 is shown a further embodiment 110 of the invention in which battery 12 is surrounded by a layer of insulation 26 to protect the battery from the thermal effects of heating pad 28 and its resistive heating element 29. In the embodiment of FIG. 3, insulation 30 is also provided outwardly of the heating pad 28 and an outer layer of foam 32 is provided as the outermost layer. It is to be appreciated that layer 32 in the embodiment of FIGS. 3 and 16 in the embodiment of FIG. 2 may be considerably thicker in a given application and as may be necessary to provide additional thermal insulation to the skin of the user.

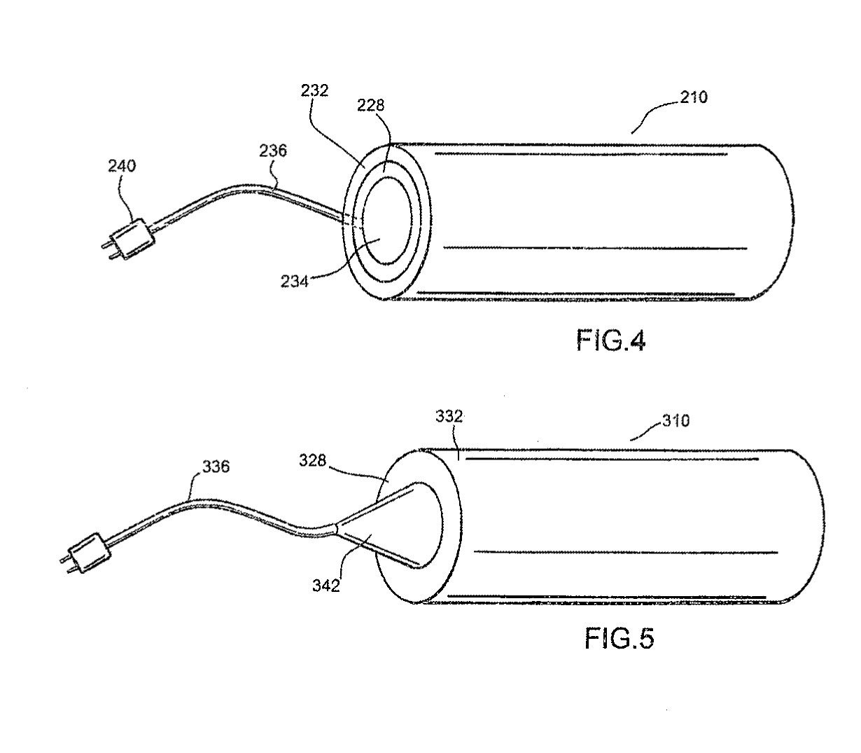

In FIG. 4 is shown an embodiment 210 of the invention in which, in lieu of the use of rechargeable battery 12 in the embodiments of 10 and 110 in FIGS. 2 and 3 respectively, an otherwise standard heating pad or thermal element is employed upon a rigid spool-like cylinder 234 which also provides structure and stability to the present thermal therapy massage roller. In FIG. 4, electrical cord 236 may be seen depending from heating pad or thermal element 228, as is electrical outlet plug 240. It is to be appreciated that a layer of insulation may be provided between heating pad 228 and a foam or gel layer 232 of the embodiment of FIG. 4. It is to be appreciated that this embodiment may also function with the use of rechargeable batteries (not shown).

Foam layer 232 may comprise various materials such as a polyurethane foam or a silicone foam, which preferably are closed-cell foams. An insulative layer may be disposed about the heating pad or thermal element 228 which, typically, would comprise a flexible insulator such as INSUL-ZONE, an EPS foam produced by Insulation Solutions of Peoria, Ill.; a polymeric aerogel; a neoprene foam; or stabilized fiberglass.

Spool cylinder 234 may be formed of a polystyrene, polycarbonate, PVC, a wood-pulp composite, or combinations thereof.

If used outdoors, a solar cell may provide power to the thermal element.

In FIG. 5 is shown a further embodiment 310 of thermal massage roller in which to cord 336 connects to heating pad 328 through an anti-kinking adapter 342 to permit roller 310 to rotate upon any part of the body without causing of any kinking or twisting of cord 336 of the system.

Through the above, there are provided various embodiments of the novel thermal therapy massage roller which may be used either by the person in need of therapy or by a professional massage person or personal massage trainer. It is to be appreciated that in the embodiments of FIGS. 2 and 3, battery 12 may be surrounded by said sleeve 115 to enhance the overall axial rigidity of the system.

While there has been shown and described above the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

I claim:

1. A thermal therapy massage roller, comprising:
(a) a cylindrical substantially rigid sleeve having an elongate cavity therein proportioned for receipt of a rechargeable battery having an electrode and charging adaptor at a base thereof;
(b) a layer of flexible insulation secured about said rigid sleeve;
(c) a pad-like circumferential heating element secured about said layer of insulation, said heating element in electrical communication with said battery; and
(d) an outer layer of a resilient partially foam-like material circumferentially surrounding said pad-like element, said foam-like material defining an outer layer of the roller.

2. The roller as recited in claim 1, further comprising:
(e) a further insulative layer secured between said pad-like heating element and said outer layer of resilient foam-like material.

3. The roller as recited in claim 1, in which said foam-like outer layer comprises a polymeric material.

4. A thermal therapy massage roller, comprising:
(a) a heat-resistant substantially rigid cylindrical core;
(b) a pad-like circumferential heating element secured about said heat resistant core, said element powered by an electrical cord having a connection to a household AC outlet;
(c) a flexible insulative layer surrounding said heating element; and
(d) a resilient foam-like outer layer surrounding said insulative layer.

5. The roller as recited in claim 4 further comprising:
a layer of insulative foam-like material surrounding said rigid core.

6. The roller as recited in Claim between said pad-like element and said electrical cord comprises:
an anti-kinking or anti-twisting adaptor.

7. The roller as recited in claim 4 in which said outer layer comprises a rubermeric material.

8. The roller as recited in claim 3, in which said foam-like outer layer is selected from the group consisting essentially of polyurethane and silicone.

9. The roller as recited in claim 3, in which said foam-like outer layer comprises a closed cell foam.

10. The roller as recited in claim 2, in which said flexible insulative layer is selected from the group consisting essentially of an EPS foam, a polymeric aerogel and neoprene.

11. The roller as recited in claim 4, in which said foam-like outer layer is selected from the group consisting essentially of polyurethane and silicone.

12. The roller as recited in claim 4, in which said foam-like outer layer comprises a closed cell foam.

13. The roller as recited in claim 4, in which said flexible insulative layer is selected from the group consisting essentially of an EPS foam, a polymeric aerogel and neoprene.
